SEQUENCE LISTING (17 sequences)

SEQ ID NO: 1 - DNA, 2079 bp, Artificial Sequence
  Puro-eGFP-Mu transposon
agatctgaagcggcgcacgaaaaacgcgaaagcgtttcacgataaatgcgaaaacggatctctgcagagctcgagtgatcatgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacatcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggatcgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcagatccgttttcgcatttatcgtgaaacgctttcgcgtttttcgtgcgccgcttcagatct

SEQ ID NO: 2 - DNA, 5068 bp, Artificial Sequence
  Puro-eGFP-pUC-Mu transposon
agatctgaagcggcgcacgaaaaacgcgaaagcgtttcacgataaatgcgaaaacggatctctgcagtgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggtcgaaaaggatcatgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacatcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggatcgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcagatccgttttcgcatttatcgtgaaacgctttcgcgtttttcgtgcgccgcttcagatct

SEQ ID NO: 3 - DNA, 54 bp, Bacteriophage Mu
gatctgaagcggcgcacgaaaaacgcgaaagcgtttcacgataaatgcgaaaac

SEQ ID NO: 4 - DNA, 20 bp, Artificial sequence
  Oligonucleotide primer
aagtgccacctgcccgatcc

SEQ ID NO: 5 - DNA, 21 bp, Artificial sequence
  Oligonucleotide primer
gtcagtagctgaacaggaggg

SEQ ID NO: 6 - DNA, 22 bp, Artificial sequence
  Oligonucleotide primer
tagcgctgatgtccggcggtgc

SEQ ID NO: 7 - DNA, 24 bp, Artificial sequence
  Oligonucleotide primer
ataggggttccgcgcacatttccc

SEQ ID NO: 8 - DNA, 20 bp, Artificial sequence
  Oligonucleotide primer
ttccacagctggttctttcc

SEQ ID NO: 9 - DNA, 20 bp, Artificial sequence
  Oligonucleotide primer
gcacttcactgacaccctca

SEQ ID NO: 10 - DNA, 20 bp, Artificial sequence
  Oligonucleotide primer
atgctttgcatacttctgcc

SEQ ID NO: 11 - DNA, 25 bp, Artificial sequence
  Oligonucleotide primer
ggggagcctggggactttccacacc

SEQ ID NO: 12 - DNA, 18 bp, Artificial sequence
  Oligonucleotide primer
atcacatggtcctgctgg

SEQ ID NO: 13 - DNA, 26 bp, Artificial sequence
  Oligonucleotide primer
cgggatcactctcggcatggacgagc

SEQ ID NO: 14 - DNA, 21 bp, Artificial sequence
  Oligonucleotide primer
tgtggaatgtgtgtcagttag

SEQ ID NO: 15 - DNA, 18 bp, Artificial sequence
  Oligonucleotide primer
gtcaggcaccgggcttgc

SEQ ID NO: 16 - DNA, 52 bp, Artificial sequence
  Oligonucleotide primer
gcgcagatctctgcagagctcgagtgatcatgtggaatgtgtgtcagttagg

SEQ ID NO: 17 - DNA, 32 bp, Artificial sequence
  Oligonucleotide primer
gcgcagatctgcggccgctttacttgtacagc

(19) United States
(12) Patent Application Publication
(10) Pub. No.: US 2010/0173800 A1
(43) Pub. Date: Jul. 8, 2010

(54) DELIVERY OF NUCLEIC ACIDS INTO GENOMES OF HUMAN STEM CELLS USING IN VITRO ASSEMBLED MU TRANSPOSITION COMPLEXES

(75) Inventor: Harri Savilahti, Helsinki (FI)

Correspondence Address: BIRCH STEWART KOLASCH & BIRCH, PO BOX 747, FALLS CHURCH, VA, 22040-0747, US

(73) Assignee: FINNZYMES OY, ESPOO (FI)

(21) Appl. No.: 12/667,853

(22) PCT Filed: Jul. 4, 2008
(86) PCT No.: PCT/FI08/50411

§ 371 Date: Feb. 17, 2010

(30) Foreign Application Priority Data
Jul. 6, 2007 (FI) 20075520

(51) Int. Cl. C40B 30/06; C12N 15/63; C07H 21/04

(52) U.S. Cl. 506/10; 435/455; 435/446; 536/23.1

(57) ABSTRACT

The present invention relates to genetic engineering and especially to the use of DNA transposition complex of bacteriophage Mu. In particular, the invention provides a gene transfer system for isolated human stem cells, wherein in vitro assembled Mu transposition complexes are introduced into a target cell and subsequently transposition into a cellular nucleic acid occurs. The invention further provides a kit for producing insertional mutations into the genomes of isolated human stem cells. The kit can be used, e.g., to generate insertional mutant libraries.

The present invention relates to genetic engineering and especially to the use of DNA transposition complex of bacteriophage Mu. In particular, the invention provides a gene transfer system for human stem cells, wherein in vitro assembled Mu transposition complexes are introduced into a target cell. Inside the cell, the complexes readily mediate integration of a transposon construct into a cellular nucleic acid. The invention further provides a kit for producing insertional mutations into the genomes of human stem cells. The kit can be used, e.g., to generate insertional mutant libraries.

### BACKGROUND OF THE INVENTION

Bacteriophage Mu replicates its genome using DNA transposition machinery and is one of the best characterized mobile genetic elements (Mizuuchi 1992; Chaconas et al., 1996). A bacteriophage Mu-derived in vitro transposition system that has been introduced by Haapa et al. (1999a) was utilised for the present invention. Mu transposition complex, the machinery within which the chemical steps of transposition take place, is initially assembled from four MuA transposase protein molecules that first bind to specific binding sites in the transposon ends. The 50 by Mu right end DNA segment contains two of these binding sites (they are called R1 and R2 and each of them is 22 by long, Savilahti et al. 1995). When two transposon ends meet, each bound by two MuA monomers, a transposition complex is formed through conformational changes. Then Mu transposition proceeds within the context of said transposition complex, i.e., protein-DNA complexes that are also called DNA transposition complexes or transpososomes (Mizuuchi 1992, Savilahti et al. 1995). Functional core of these complexes are assembled from a tetramer of MuA transposase protein and Mu-transposon-derived DNA-end-segments (i.e. transposon end sequences recognised by MuA) containing MuA binding sites. When the core complexes are formed they can react in divalent metal ion-dependent manner with any target DNA and insert the Mu end segments into the target (Savilahti et al 1995). A hallmark of Mu transposition is the generation of a 5-bp target site duplication (Allet, 1979; Kahmann and Kamp, 1979).

In the simplest case, the MuA transposase protein and a short 50 by Mu right-end (R-end) fragment are the only macromolecular components required for transposition complex assembly and function (Savilahti et al. 1995, Savilahti and Mizuuchi 1996). Analogously, when two R-end sequences are located as inverted terminal repeats in a longer DNA molecule, transposition complexes form by synap sing the transposon ends. Target DNA in the Mu DNA in vitro transposition reaction can be linear, open circular, or supercoiled (Haapa et al. 1999a).

To date Mu in vitro transposition-based strategies have been utilized efficiently for a variety of molecular biology applications including DNA sequencing (Haapa et al. 1999a; Butterfield et al. 2002), generation of DNA constructions for gene targeting (Vilen et al., 2001), and functional analysis of plasmid and viral (HIV) genomic DNA regions (Haapa et al., 1999b, Laurent et al., 2000). Also, functional genomics studies on whole virus genomes of potato virus A and bacteriophage PRD1 have been conducted using the Mu in vitro transposition-based approaches (Kekarainen et al., 2002, Vilen et al., 2003). In addition, pentapeptide insertion mutagenesis method has been described (Taira et al., 1999, Poussu et al., 2004). An insertional mutagenesis strategy for bacterial genomes has also been developed in which the in vitro assembled functional transpososomes were delivered into various bacterial cells by electroporation (Lamberg et al., 2002).

E. coli is the natural host of bacteriophage Mu. It was first shown with E. coli that in vitro preassembled transposition complexes can be electroporated into the bacterial cells whereby they then integrate the transposon construct into the genome (Lamberg et al., 2002). The Mu transpososomes were also able to integrate transposons into the genomes of three other Gram negative bacteria tested, namely, Salmonella enterica (previously known as S. typhimurium), Erwinia carotovara, and Yersinia enterocolitica (Lamberg et al. 2002). In each of these four bacterial species the integrated transposons were flanked by a 5-bp target site duplication, a hallmark of Mu transposition, thus confirming that the integrations were generated by DNA transposition chemistry. Essentially same results were also obtained with gram-negative bacteria (Pajunen et al., 2005). Finally, it was disclosed in WO 2004/090146 that eukaryotic cells, such as mammalian cells, can be transfected with this method.

Other currently existing gene transfer systems for mammalian cells are based on virus vectors, naked DNA, or DNA-carrier complexes. Although widely used, they each have their limitations (Thomas et al., 2003; Wiethoff and Middaugh, 2003). There can be problems connected with safety and efficiency as well as difficulties in preparing large quantities of the vector. Also concatemerization of the integrated transgene at the insertion locus can be a disadvantage in some applications, as multiple copies of the transgene will be integrated. Host range may also be limited to certain cell types only. The Mu-based system does not have the safety risks associated with viral vectors such as lentiviral vectors (Gropp et al., 2003), and it is relatively cost-efficient and easy to handle. Importantly, strong viral promoters are avoided, further emphasizing the safety aspect particularly when transfecting human cells such as human stem cells.

### SUMMARY OF THE INVENTION

The present invention discloses a gene transfer system for human stem cells that utilizes in vitro-assembled phage Mu DNA transposition complexes. Linear DNA molecules containing appropriate selectable markers and other genes of interest are generated that are flanked by DNA sequence elements needed for the binding of MuA transposase protein. Incubation of such DNA molecules with MuA protein results in the formation of DNA transposition complexes, transpososomes. These can be delivered into human stem cells by electroporation or by other related methods. The method described in the present invention expands the applicability of the Mu transposon as a gene delivery vehicle into human stem cells.

In a first aspect, the invention provides a method for incorporating nucleic acid segments into cellular nucleic acid of an isolated human stem cell, the method comprising the step of:

delivering into the human stem cell a Mu transposition complex that comprises (i) MuA transposases and (ii) a transposon segment that comprises a pair of Mu end sequences recognised and bound by MuA transposase and an insert sequence between said Mu end sequences, preferably under conditions that allow integration of the transposon segment into the cellular nucleic acid.

In another aspect, the invention features a method for forming an insertion mutant library from a pool of isolated human stem cells, the method comprising the steps of:

a) delivering into a human stem cell a Mu transposition complex that comprises (i) MuA transposases and (ii) a transposon segment that comprises a pair of Mu end sequences recognised and bound by MuA transposase and an insert sequence with a selectable marker between said Mu end sequences, preferably under conditions that allow integration of the transposon segment into the cellular nucleic acid,
b) screening for cells that comprise the selectable marker.

In a third aspect, the invention provides a kit for incorporating nucleic acid segments into cellular nucleic acid of a human target cell such as human stem cell.

The term “transposon”, as used herein, refers to a nucleic acid segment, which is recognised by a transposase or an integrase enzyme and which is essential component of a functional nucleic acid-protein complex capable of transposition (i.e. a transpososome). Minimal nucleic acid-protein complex capable of transposition in the Mu system comprises four MuA transposase protein molecules and a transposon with a pair of Mu end sequences (e.g. SEQ ID NO:3) that are able to interact with MuA.

The term “transposase” used herein refers to an enzyme, which is an essential component of a functional nucleic acid-protein complex capable of transposition and which is mediating transposition. The term “transposase” also refers to integrases from retrotransposons or of retroviral origin.

The expression “transposition” used herein refers to a reaction wherein a transposon inserts itself into a target nucleic acid. Essential components in a transposition reaction are a transposon and a transposase or an integrase enzyme or some other components needed to form a functional transposition complex. The gene delivery method and materials of the present invention are established by employing the principles of in vitro Mu transposition (Haapa et al. 1999ab and Savilahti et al. 1995).

The term “transposon end sequence” used herein refers to the conserved nucleotide sequences at the distal ends of a transposon. The transposon end sequences are responsible for identifying the transposon for transposition.

The term “human stem cells”, as used herein, refers to unspecialized human cells capable of dividing and renewing themselves for long periods and giving rise to specialized cell types. Particularly, the term “human stem cells” refers to embryonic stem cells and adult stem cells. Human embryonic stem (hES) cells are pluripotent cells derived from the inner cell mass of the early preimplantation embryo. Another group of human stem cells are those originating from umbilical cord blood. Recently, it has been shown that pluripotent human stem cells can be induced from adult human somatic cells such as fibroblasts (Takahashi & Yamanaka, 2007; Wernig et al 2007; Yu et al, 2007). The present invention is also directed to the modification of these induced pluripotent stem (iPS) cells.

Human adult stem cells, i.e. somatic stem cells, are undifferentiated cells found among differentiated cells in a tissue or organ. Human adult stem cells can renew themselves, and can differentiate to yield the major specialized cell types of the tissue or organ. Examples of human adult stem cells are hematopoietic stem cells, neural stem cells, epithelial stem cells, skin stem cells and bone marrow stromal cells. Both embryonic stem cells and adult stem cells can be grown in a laboratory as a cell line culture. The present invention is preferably directed to the transformation of human stem cells grown as laboratory cell lines. The hES cells used in the present method are preferably obtained from currently known human stem cell lines grown in laboratory conditions. Further, these human stem cell lines are preferably listed in The NIH Human Embryonic Stem Cell Registry (National Institutes of Health, 9000 Rockville Pike, Bethesda, Md. 20892, USA; see also http://stemcells.nih.gov/research/registry/).

### BRIEF DESCRIPTION OF THE DRAWINGS

FIGS. 1A and 1B. 1A, The schematic outline of the use of the transposon as a gene transfer vector. First, the transposon DNA and a tetramer of MuA transposase assemble into a stable protein-DNA complex, transpososome. The presence of Mg²⁺ ions in vivo activates the transpososome, which then mediates the integration of the transposon into human chromosomal DNA. 1B, Puro-eGFP-Mu and Puro-eGFP-pUC-Mu transposons. The marker genes and the promoters and terminators are marked below the transposons. The gray boxes at the ends of the transposons indicate the MuA binding site.

FIGS. 2A and 2B. Southern blot analysis of the insertions into the human cell genomes. 2A. Genomic DNA of G418-resistant HeLa cell clones was digested with BamHI+BglII and probed with the Kan/Neo-p15A-Mu transposon DNA. Transposon insertion mutants (lanes 1-17), genomic DNA of original HeLa cell strain as a negative control (C), HeLa cell genomic DNA plus transposon DNA as a positive control (P). The sizes of marker (M) fragments are shown on the right. 2B. Genomic DNA of puromycin-resistant human ES cells was digested with BglII or EcoRI and probed with Puro-eGFP-Mu transposon DNA.

### DETAILED DESCRIPTION OF THE INVENTION

The in vitro assembled Mu transposition complex is stable but catalytically inactive in conditions devoid of Mg²⁺ or other divalent cations (Savilahti et al., 1995; Savilahti and Mizuuchi, 1996). After electroporation into target cells, these complexes remain functional and become activated for transposition chemistry upon encountering Mg²⁺ ions within the cells, facilitating transposon integration into host chromosomal DNA (Lamberg et al., 2002). The in vitro preassembled transpososomes do not need special host cofactors for the integration step in vivo (Lamberg et al., 2002). Importantly, once introduced into cells and integrated into the genome, the inserted DNA will remain stable in cells that do not express MuA (Lamberg et al., 2002).

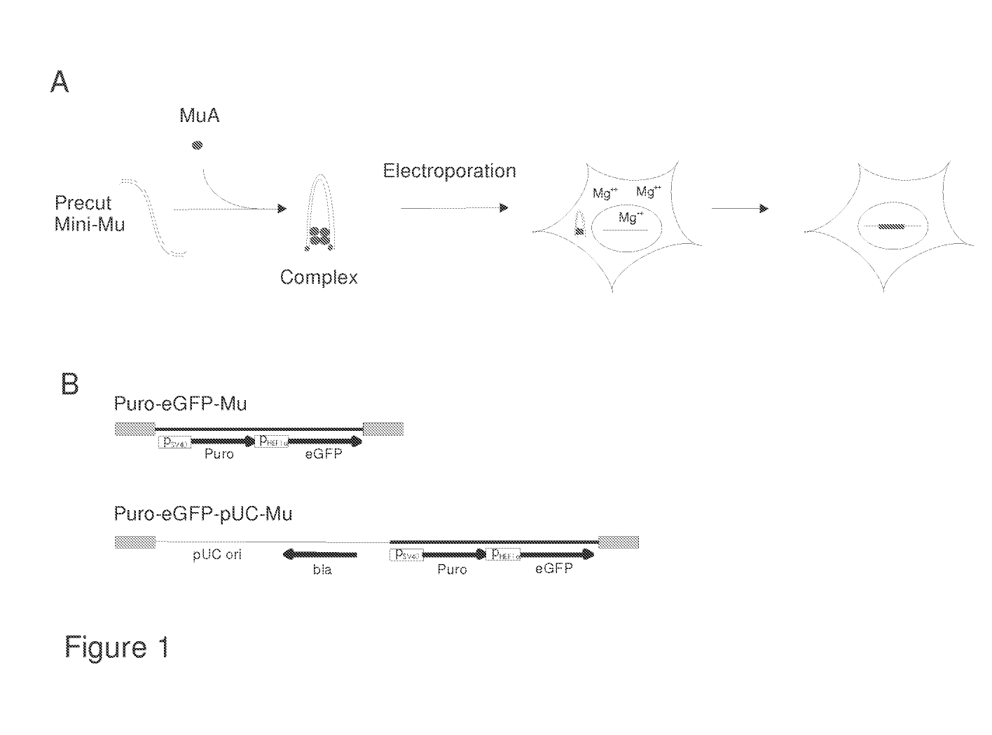

To study if the Mu transposition system with the in vitro assembled transpososomes works also for human cells, particularly human stem cells, we constructed transposons (antibiotic resistance markers connected to Mu ends, see FIGS. 1A and 1B), assembled the complexes and tested the transposition strategy. Transposon integration sites were determined after electroporation following propagation of target cells on selective growth medium. The transposons were integrated into the genomes with a 5-bp target site duplication flanking the insertion, indicating that a genuine DNA transposition reaction had occurred. These results demonstrate that, surprisingly, the conditions in human stem cells allow the integration of Mu DNA. Remarkably, the nuclear membrane, DNA binding proteins, or DNA modifications or conformations did not prevent the integration. Furthermore, the structure and catalytic activity of the Mu complex retained even after a concentration step. This expands the applicability of the Mu transposition strategy into human stem cells. The benefit of this system is that there is no need to generate an expression system of the transposition machinery for the organism of interest.

The efficient strategy for stable genetic modification of human stem cells, such as hES cells, provided by the present invention is highly valuable for manipulating the cells in vitro and promotes the study of human stem cell biology, human embryogenesis, and the development of cell-based therapies. In general, human stem cells include human embryonic stem cells and cells derived from human embryonic stem cells that have retained a capacity to differentiate towards a particular cell type. Human stem cell populations include those involved in producing neuronal cells, muscle cells, blood cells etc.

The invention provides a method for incorporating nucleic acid segments into cellular nucleic acid of an isolated human stem cell or a group of such cells (such as a cell culture), the method comprising the step of:

delivering into the human stem cell an in vitro assembled Mu transposition complex that comprises (i) MuA transposases and (ii) a transposon segment that comprises a pair of Mu end sequences recognised and bound by MuA transposase and an insert sequence between said Mu end sequences, preferably under conditions that allow integration of the transposon segment into the cellular nucleic acid.

For the method, one can assemble in vitro stable but catalytically inactive Mu transposition complexes in conditions devoid of Mg²⁺ as disclosed in Savilahti et al., 1995 and Savilahti and Mizuuchi, 1996. In principle, any standard physiological buffer not containing Mg²⁺ is suitable for the assembly of said inactive Mu transposition complexes. However, a preferred in vitro transpososome assembly reaction may contain 150 mM Tris-HCl pH 6.0, 50% (v/v) glycerol, 0.025% (w/v) Triton X-100, 150 mM NaCl, 0.1 mM EDTA, 55 nM transposon DNA fragment, and 245 nM MuA. The reaction volume may be for example 20 or 80 microliters. The reaction is incubated at about 30° C. for 0.5-4 h, preferably 2 h. To obtain a sufficient amount of transposition complexes for delivery into the cells, the reaction is then concentrated and desalted from several assembly reactions. For the transformations the final concentration of transposition complexes compared to the assembly reaction is preferably at least 8-fold, more preferably 10-fold, and most preferably at least 20-fold. The concentration step is preferably carried out by using centrifugal filter units. Alternatively, it may be carried out by centrifugation or precipitation (e.g. using PEG or other types of precipitants).

In the method, the concentrated transposition complex fraction is delivered into the human target cell. The preferred delivery method is electroporation. The electroporation of Mu transposition complexes into bacterial cells is disclosed in Lamberg et al., 2002. However, the method of Lamberg et al. cannot be directly employed for introduction of the complexes into eukaryotic cells. A variety of DNA introduction methods are known for eukaryotic cells and the one skilled in the art can readily utilize these methods in order to carry out the method of the invention (see e.g. Sands and Hasty, 1997; “Electroporation Protocols for Microorganisms”, ed. Jac A. Nickoloff, Methods in Molecular Biology, volume 47, Humana Press, Totowa, N.J., 1995; “Animal Cell Electroporation and Electrofusion Protocols”, ed. Jac A. Nickoloff, Methods in Molecular Biology, volume 48, Humana Press, Totowa, N.J., 1995; and “Plant cell Electroporation and Electrofusion Protocols”, ed. Jac A. Nickoloff, Methods in Molecular Biology, volume 55, Humana Press, Totowa, N.J., 1995). Such DNA delivery methods include direct injections by the aid of needles or syringes, exploitation of liposomes, and utilization of various types of transfection-promoting additives. Physical methods such as particle bombardment may also be feasible.

Transposition into the cellular nucleic acid of the target cell seems to follow directly after the electroporation without additional intervention. However, to promote transposition and remedy the stress caused by the electroporation, the cells can be incubated at about room temperature to 30° C. for 10 min-48 h or longer in a suitable medium before plating or other subsequent steps. Preferably, a single insertion into the cellular nucleic acid of the target cell is produced.

The insert sequence between Mu end sequences preferably comprises a selectable marker, gene or promoter trap or enhancer trap constructions, protein expressing or RNA producing sequences. Preferably said marker for human cells is the pac gene allowing puromycin selection. Such constructs renders possible the use of the method in gene tagging, functional genomics or gene therapy.

The term “selectable marker” above refers to a gene that, when carried by a transposon, alters the ability of a cell harboring the transposon to grow or survive in a given growth environment relative to a similar cell lacking the selectable marker. The transposon nucleic acid of the invention preferably contains a positive selectable marker. A positive selectable marker, such as an antibiotic resistance, encodes a product that enables the host to grow and survive in the presence of an agent, which otherwise would inhibit the growth of the organism or kill it. The insert sequence may also contain a reporter gene, which can be any gene encoding a product whose expression is detectable and/or quantitatable by immunological, chemical, biochemical, biological or mechanical assays. A reporter gene product may, for example, have one of the following attributes: fluorescence (e.g., green fluorescent protein), enzymatic activity (e.g., luciferase, lacZ/β-galactosidase), toxicity (e.g., ricin) or an ability to be specifically bound by a second molecule (e.g., biotin). The use of markers and reporter genes in eukaryotic cells, such as human cells, is well-known in the art.

Since the target site selection of in vitro Mu system is known to be random or nearly random, one preferred embodiment of the invention is a method, wherein the nucleic acid segment is incorporated to a random or almost random position of the cellular nucleic acid of the target cell. However, targeting of the transposition can be advantageous in some cases and thus another preferred embodiment of the invention is a method, wherein the nucleic acid segment is incorporated to a targeted position of the cellular nucleic acid of the target cell. This could be accomplished by adding to the transposition complex, or to the DNA region between Mu ends in the transposon, a targeting signal on a nucleic acid or protein level. Said targeting signal is preferably a nucleic acid, protein or peptide which is known to efficiently bind to or associate with a certain nucleotide sequence, thus facilitating targeting.

One specific embodiment of the invention is the method wherein a modified MuA transposase is used. Such MuA transposase may be modified, e.g., by a deletion, an insertion or a point mutation and it may have different catalytic activities or specifities than an unmodified MuA.

Another embodiment of the invention is a method for forming an insertion mutant library from a pool of isolated human stem cells, the method comprising the steps of:

a) delivering into a human stem cell an in vitro assembled Mu transposition complex that comprises (i) MuA transposases and (ii) a transposon segment that comprises a pair of Mu end sequences recognised and bound by MuA transposase and an insert sequence with a selectable marker between said Mu end sequences, preferably under conditions that allow integration of the transposon segment into the cellular nucleic acid.
b) screening for cells that comprise the selectable marker.

In the above method, a person skilled in the art can easily utilise different screening techniques. The screening step can be performed, e.g., by methods involving sequence analysis, nucleic acid hybridisation, primer extension or antibody binding. These methods are well-known in the art (see, for example, Current Protocols in Molecular Biology, eds. Ausubel et al, John Wiley & Sons: 1992). Libraries formed according to the method of the invention can also be screened for genotypic or phenotypic changes after transposition.

Further embodiment of the invention is a kit or use of a kit for incorporating nucleic acid segments into cellular nucleic acid of a human stem cell. The kit comprises a concentrated fraction of Mu transposition complexes that comprise a transposon segment with a marker, which is selectable in human stem cells. Preferably, said complexes are provided as a substantially pure preparation apart from other proteins, genetic material, and the like.

The publications and other materials used herein to illuminate the background of the invention, and in particular, to provide additional details with respect to its practice, are incorporated herein by reference. The invention will be described in more detail in the following Experimental Section.

### Experimental Section

### Strains and Media

HeLa cells were maintained in modified Eagle's medium (MEM, Gibco, Carlsbad, Calif., USA) supplemented with 10% foetal calf serum (European origin, Autogen Bioclear), 50 U/ml penicillin, 50 μg/ml streptomycin (100× Penicillin-streptomycin, Gibco) and 2 mM L-glutamine (Gibco) at 37° C. and 5% CO₂ in a humidified tissue culture incubator. Selective conditions consisted of 400 μg/ml G418 for HeLa cells.

The isolation of FES 29 embryonic stem cell line is described in Mikkola, M. et al. 2006. Human FES 29 embryonic stem cells were maintained on MEF feeders as described (Mikkola, M. et al. 2006). MEF feeders (mitotically inactivated by Mitomycin-C, density 10 000 cells/cm²) in serum-free medium (KnockoutD-MEM; Invitrogen, Paisley, UK) supplemented with 2 mM L-Glutamin/Penicillin streptomycin (Sigma-Aldrich), 20% Knockout Serum Replacement (Gibco), 1× non-essential amino acids (Gibco), 0.1 mM betamercaptoethanol (Gibco), 1×ITS (Sigma-Aldrich) and 4 ng/ml recombinant bFGF (Invitrogen).

### Enzymes and Reagents

Wild type MuA transposase (MuA) and proteinase K were obtained from Finnzymes, Espoo, Finland. Restriction endonucleases and the plasmid pUC19 were from New England Biolabs, a Klenow enzyme was from Promega. Enzymes were used as recommended by the suppliers. Bovine serum albumin and heparin were from Sigma. [α³²P]dCTP (1000-3000 Ci/mmol) was f₁ Amersham Biosciences. Mutant E392Q MuA transposase (Baker & Luo, 1994) was purified as described in (Baker et al., 1993). See Table 2 for primers used in this study.

### Standard DNA Techniques

Plasmid DNA from E. coli was isolated using purification kits from Qiagen, as recommended by the supplier. Standard DNA manipulation and cloning techniques, including PCR for plasmid engineering, were performed as described by (Sambrook & Russell, 2001), and DNA-modifying enzymes were used as recommended by the suppliers. DNA sequence determination was performed at the DNA sequencing facility of the Institute of Biotechnology (University of Helsinki).

### Transposons

The mini-Mu transposons (FIG. 1B) were isolated by BglII digestion from their respective carrier plasmids. The DNA fragment was purified chromatographically as described (Haapa et al. 1999a).

### Construction of Kan/Neo-Mu Transposon

A neomycin-resistance cassette containing a bacterial promoter, SV40 early promoter, kanamycin/neomycin resistance gene, and Herpes simplex virus thymidine kinase polyadenylation signals was generated by PCR from pIRES2-EGFP plasmid (Clontech). After addition of Mu end sequences using standard PCR-based techniques, the construct was cloned as a BglII fragment into a vector backbone derived from pUC19. The construct was confirmed by DNA sequencing.

### Construction of Puro-eGFP-Mu Transposons

SV40-Puro fragment was amplified by PCR from the retrovirus vector pBABEPuro (Morgenstern & Land, 1990; Addgene plasmid 1764), 5′ phosphates were added, and the fragment was ligated to EcoRV site of the plasmid pSIN18.cPPT.hEF-1α.EGFP.WPRE (Gropp et al. 2003). To generate Puro-eGFP-Mu transposon SV40-Puro-hEFa1-EGFP fragment was amplified by PCR, digested with BglII, and ligated to the Cat-Mu transposon carrier plasmid (Haapa et al. 1999b) BamHI fragment replacing the cat gene. Puro-eGFP-pUC-Mu transposon was generated by cloning pUC19 sequence into the Puro-eGFP-Mu transposon.

### In Vitro Transpososome Assembly

The in vitro transpososome assembly was performed essentially as described previously (Lamberg, A. 2002). The in vitro transpososome assembly reaction (80 μl) contained 55 nM transposon DNA fragment, 245 nM MuA, 150 mM Tris-HCl pH 6.0, 50% (v/v) glycerol, 0.025% (w/v) Triton X-100, 150 mM NaCl, 0.1 mM EDTA. The reaction was carried out at 30° C. for 2-6 h. The complex was concentrated and desalted from several reactions approximately tenfold by Centricon YM-100 centrifugal cartridge (100 kDa cut-off; Millipore) as described previously (Pajunen et al., 2005) or alternatively by PEG (polyethylene glycol)-precipitation essentially as described for bacterial viruses by Savilahti and Bamford (1993). The assembly and concentration of transpososomes was monitored by agarose/BSA/heparin gels as described previously (Lamberg et al., 2002).

### Electroporation

Growing human HeLa cells were harvested with trypsin-EDTA, pH 7.4 (Gibco) and washed once, twice or three times with 1×PBS (137 mM NaCl, 2.7 mM KCl, 4.3 mM Na₂HPO₄, 1.47 mM KH₂PO₄). Mortality of harvested cells was determined by trypan blue inclusion: trypan blue (AppliChem) was added to a final concentration of 0.2% and the amount of living and dead cells were counted with the help of a hemocytometer. The cells were subsequently resuspended in 1×PBS. Unless otherwise specified, standard electroporation conditions were: 1-4×10⁶ HeLa cells in 800 μl of 1×PBS, and 2-3 μg of DNA. The cells were exposed to a single voltage pulse (250 V 500 μF) at room temperature, allowed to remain in the cuvette for ten minutes, and the plated onto tissue culture dishes. Selection was initiated 48 hr after electroporation and G418-resistant colonies were obtained after 10 days selection. After selection, colonies were fixed with cold methanol, stained with 0.2% methylene blue, air-dried, and counted.

Human ES cells were detached either with 200 units/ml collagenase IV (Gibco) for 5-10 min at 37° C. (whereafter the cells were scraped and dissociated by gently pipetting), or with 1× Tryple™ (GIBCO) for 3 min at RT and resuspended in Ca2+/Mg2+ free PBS or standard hESC culture medium. 3.3 μg of transpososomes were mixed with the 800 μl of cells (approximately 1-4×10⁶ cells) in a cold 0.4 cm cuvette and given immediately a single voltage pulse (320 V, 500 μl or 250 V, 100 μl). After 2 min incubation RT medium was added and the cells plated on feeder cells. Puromycin selection was started 3-5 days after the electroporation. Electroporated cells were selected for 2 days with 1 μg/ml puromycin (Sigma). The cells were then cultured up to confluent, passaged on new plates, cultured for 3 days and selected again for 2 days with 1 μg/ml puromycin.

### Cell Cloning

Following electroporation of HeLa cells, pure integrant clones were obtained by picking separate colonies, which were detached from the plate by scraping with a pipette tip, trypsinised in a well of a 96-well plate, and plated on a gelatinised well. The clones were grown and plated again so that single cells were widely scattered on the plate. After cells had attached on to the plate, single, well separated cells were marked on the bottom of the plate. When the colonies had grown enough, these marked colonies were picked up and propagated.

### Isolation of the Genomic DNA

HeLa and ES cells were collected from 10 cm culture plates and suspended in 5 ml of the proteinase K digestion buffer (10 mM Tris-HCl (pH 8.0), 400 mM NaCl, 10 mM EDTA, 0.5% SDS, and 200 μg/ml proteinase K). The proteinase K treatment was carried out at 55° C. until no cells were visible. When necessary, more proteinase K was added. Following the proteinase K treatment, 1.5 ml of 6 M NaCl was added followed by centrifugation (20 min, 8.5 K). The supernatant was collected and precipitated with ethanol. RNA was removed by RNaseA treatment (100 μg/ml). The DNA was extracted once with phenol:chloroform:isoamylalcohol (25:24:1, by vol.) and once with chloroform:isoamylalcohol (24:1, v/v), precipitated and dissolved in TE (10 mM Tris-HCl, pH 8.0 and 1 mM EDTA).

### Southern Blotting

For blotting, genomic DNA was digested with restriction enzymes. The fragments were separated on a 0.8% agarose gel (Seakem LE). The DNA was transferred with 20×SSC to a nylon filter (Hybond-N+, Amersham) and fixed with UV light (Stratalinker UV cross-linker; Stratagene) or transferred with 0.4 M NaOH without the UV fixing. Southern hybridization was carried out essentially as described in Sambrook & Russell, 2001, with [α³²P]dCTP-labeled (Random Primed, Roche or Rediprime II Random Prime, GE Healthcare) probes. Visualization was done by autoradiography using the Fujifilm Image Reader BAS-1500 or Fuji FLA-5000.

### Determination of Transposon Location

Cloning. Genomic DNA of G418-resistant HeLa cells was digested with one or two restriction enzymes that did not cut the transposon. The fragments with a transposon attached to its chromosomal DNA flanks were either cloned into pUC19 selecting for kanamycin and ampicillin resistance or self-ligated selecting for kanamycin resistance. DNA sequences of transposon borders were determined from these plasmids using transposon specific primers. Genomic locations were identified using the BLAST search at Ensembl Genome Browser (http://www.ensembl.org/index.html), SDSC Biology WorkBench (http://workbench.sdsc.edu/), or NCBI (http://www.ncbi.nlm.nih.gov/).

Inverse PCR. Genomic DNA from puromycin-resistant ES cells was digested with a combination of restriction enzymes (NheI+SpeI+XbaI; DraI+HpaI+SnaBI) producing compatible ends but not cutting the transposon, and the restriction fragments generated were self-ligated. The ligation reactions were used as templates in nested PCR reactions with transposon specific primers. DNA sequences of transposon borders and the genomic location of the insertion were determined as above.

### Results

Gene transfer techniques are an essential tool for genomics studies with varying demands for different types of cells from different organisms. A variety of techniques are available for a number of cells. However, no general strategy is available for eukaryotic cells. Phage Mu transposition system can be modified for a variety tasks including applications as a gene transfer vector. Our previous success of mutagenizing both gram-negative and gram-positive bacteria prompted us to test the system also in eukaryotic cells. FIG. 1A shows the overall strategy used for transfection.

The transposons used for bacteria contained a selectable marker between the 50 by of DNA derived from the Mu R-end. For the human ES cells we constructed a Puro-eGFP-Mu transposon (SEQ ID NO:1) with puromycin resistance gene under SV40 promoter and eGFP gene under human EF1α promoter between the Mu ends and a Puro-eGFP-pUC-Mu transposon (SEQ ID NO:2) with pUC19 inserted in the transposon (FIG. 1B).

Mu transpososomes assembled in the absence of divalent metal ions are catalytically inert but very stable. We assembled Mu transpososomes by incubating the precut transposons with MuA, and concentrated the assembly products approximately ten-fold (see Table 3). Analytical gel retardation assay verified successful assembly and concentration of transpososomes (not shown).

Integration of the Transposon into the Human Genome

Having established an efficient system in other cell types, we wanted to ascertain its functionality also in human cells. The HeLa cell is an immortal cell line used widely in medical research and thus was the first choice as the model for human cells. The HeLa cells were electroporated with pre-assembled, concentrated transpososomes, and the controls included transpososomes assembled with inactive MuA E392Q mutant as well as the linear transposon-DNA as such. The transfected cells were selected on the basis of the G418 resistance. Human ES cells have great potential to be used for gene therapy and thus are an important target for genomics research. The hES cells were electroporated with pre-assembled, concentrated transpososomes. The transfected cells were selected on the basis of the puromycin resistance.

We determined the transfection efficiency of the HeLa cells as colony forming units per microgram of DNA used in electroporation and the transfection rate as the percentage of the surviving cells that were transfected. The active transpososomes yielded about 2400 cfu/μg DNA compared to about 40 cfu/μg DNA for the inactive mutant complexes and about 100 cfu/μg DNA for the linear transposon. Thus, the transpososomes enhanced the transfection efficiency about 20-fold as compared to the linear transposon or about 60-fold as compared to the inactive transpososomes. The transfection rate was about 0.2% of the cells that survived the electroporation.

The corresponding transfection efficiency of the hES cells in electroporation (320 V, 500 μF) of 3.1×10⁶ cells with 5 μg of DNA was ˜11 000 resistant colony forming units with the transposon complex and ˜300 resistant colony forming units with the linear transposon DNA (i.e. control DNA).

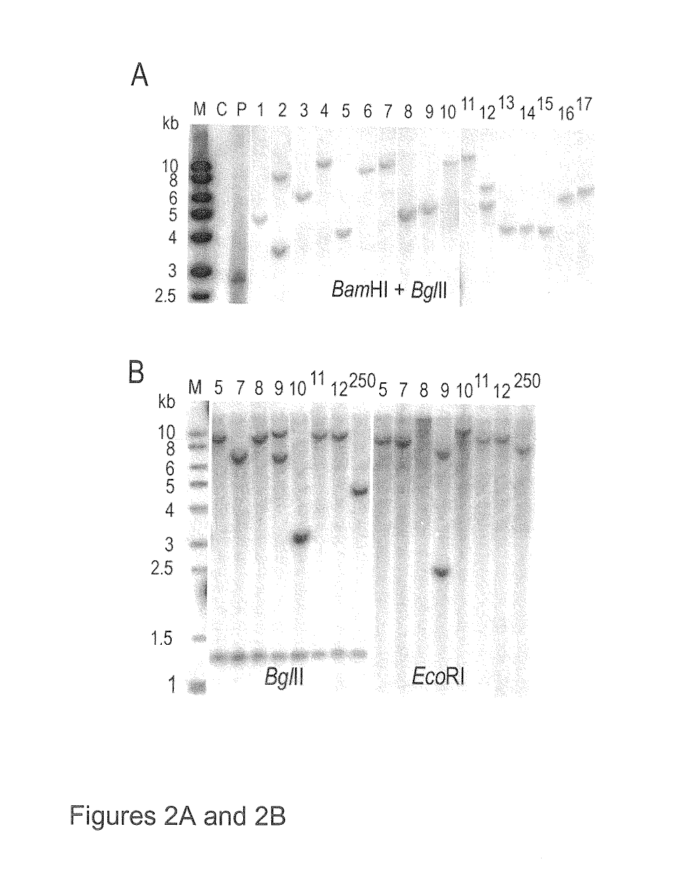

To study the copy number of the integrated transposon in the human cells we performed Southern blot analysis with HeLa and hESC clones (FIGS. 2A and 2B). The genomic DNA of the resistant HeLa clones was digested with BamHI and BglII that do not cut the transposon-DNA. Using the transposon as a probe we got a positive result with all the clones analysed, and we also detected more than one band in about 10% of the analysed clones. The result suggests that about 90% of the obtained HeLa clones contained one integrated transposon.

The genomic DNA of the resistant hESC clones was digested with EcoRI and BglII, that do not cut the transposon-DNA. Using the transposon as a probe we got a positive result with all the clones analysed, i.e. transposon integrations can be seen as a band in a blot (see FIG. 2B). One of the clones had two bands indicating possibly double integration.

### The Location of Insertions in the Human Genome

As the Mu transposition produces a 5 by duplication at the insertion site we analysed the clones by sequencing to verify that the resistant clones are the products of a true transposition reaction. The integrations were localized in the human genome using Ensembl Genome Browser. The flanking sequences and the classification of the integrations are shown in Table 1.

TABLE 1

| | | Chromo- | | | |
|---|---|---|---|---|---|
| Clone | Genomic Sequence | some | Band | Position | Gene(s)/* |
| HeLa cells | | | | | |
| RGC16 | aggaggaagaACCAG(Kan/Neo-LoxP-Mu) | 8 | q24.21 | 12836325-29 | FAM84B-MYC |
| | ACCAGgcacatgctg | | | | |
| RGC26 | ttaaatgaacTTCAG(Kan/Neo-LoxP-Mu) | 12 | p12.3 | 15381980-84 | PTPRO_HUMAN/Intron/+ |
| | TTCAGgaaaataatg | | | | |
| RGC35 | ttgttcagttCTGGT(Kan/Neo-LoxP-Mu) | 2 | q31.2 | 179679743.47 | NP_775919.2-SESTD1 |
| | CTGGTgactcattgg | | | | |
| RGC200.1A | agggggatccCCGGC(Kan/Neo-p15A-Mu) | 5 | q35.3 | 179178676-80 | MGAT4B-SQSTM1 |
| | CCGGCccctgctgcc | | | | |
| RGC204.1B | ttgagtcaagAGGGG(Kan/Neo-p15A-Mu) | 1 | c21.3 | 149586575-79 | ENSESTG00000020135/Intron/+ |
| | AGGGGgaagtccggg | | | | |
| RGC205.1A | aagcatcaggCTGGG(Kan/Neo-p15A-Mu) | 1 | p36.13 | 16855907-11 | Q49A61_HUMAN-729574 |
| | CTGGTcaggtggagg | | | | |
| RGC209.1F | cccagacttcACCAT(Kan/Neo-p15A-Mu) | 1 | q21.3 | 152313986-90 | Nup210L/Intron/+ |
| | ACCATtgtgtcatac | | | | |
| RGC210.1A | caacaatttcATAGG(Kan/Neo-p15A-Mu) | 20 | q12 | 38737377-81 | RP1-191L6.2-001-MAFB |
| | ATAGGgttcagccta | | | | |
| RGC214.1A | ttgcagtgagCCGAG(Kan/Neo-p15A-Mu) | 5 | q13.3 | 75118286-90 | NP_001013738.1-SV2 |
| | CCGAGatcctgccac | | | | |
| Human ES cells | | | | | |
| 4 | ttgcccaggcTGGAG(Puro-eGFP-Mu)TGG | 1 | p34.3 | 36223437-41 | EIF2C3/Intron/− |
| | AGtacagtggct | | | | |
| 8 | agccaccgcgCCCGG(Puro-eGFP-Mu)CCC | 5 | q31.1 | 133903082-86 | PHF15/Intron/+ |
| | GGccaatcctgg | | | | |
| 9 | tcttcaaataGAGAT(Puro-eGFP-Mu)GAG | 18 | p11.1 | 5408820-24 | EPB41L3/Intron/+ |
| | ATggagaatcac | | | | |
| 12 | tgtaactcacCCCTG(Puro-eGFP-Mu)CCC | 17 | q25.3 | 72973536-40 | SEPT9/Intron/+ |
| | TGgaaggaggct | | | | |
| 250 | ggctactgtgGGCAC(Puro-eGFP-Mu)GGC | 3 | q25.1 | 152372945-49 | MED12L/Intron/+ |
| | ACacacagatac | | | | |

*, + transposon parallel with the gene, −, opposite direction

TABLE 2
Primers used in this study.

| Oligonucleotide | Comment | Sequence 5′-3′ |
|---|---|---|
| HSP-520 | Sequencing (Kan/Neo) | AAGTGCCACCTGCCCGATCC |
| | | SEQ ID NO: 4 |
| HSP-521 | Sequencing (Kan/Neo) | GTCAGTAGCTGAACAGGAGGG |
| | | SEQ ID NO: 5 |
| HSP-550 | Sequencing (Kan/Neo) | TAGCGCTGATGTCCGGCGGTGC |
| | | SEQ ID NO: 6 |
| HSP-551 | Sequencing (Kan/Neo) | ATAGGGGTTCCGCGCACATTTCCC |
| | | SEQ ID NO: 7 |
| HSP-563 | Sequencing (Kan/Neo) | TTCCACAGCTGGTTCTTTCC |
| | | SEQ ID NO: 8 |
| HSP-564 | Sequencing (Kan/Neo) | GCACTTCACTGACACCCTCA |
| | | SEQ ID NO: 9 |
| HSP-565 | Inverse PCR (Puro-GFP) | ATGCTTTGCATACTTCTGCC |
| | | SEQ ID NO: 10 |
| HSP-566 | Inverse PCR and sequencing (Puro-GFP) | GGGGAGCCTGGGGACTTTCCACACC |
| | | SEQ ID NO: 11 |
| HSP-567 | Inverse PCR (Puro-GFP) | ATCACATGGTCCTGCTGG |
| | | SEQ ID NO: 12 |
| HSP-568 | Inverse PCR and sequencing (Puro-GFP) | CGGGATCACTCTCGGCATGGACGAGC |
| | | SEQ ID NO: 13 |
| Puro f2 | PCR primer (Puro) | TGTGGAATGTGTGTCAGTTAG |
| | | SEQ ID NO: 14 |
| Puro r2 | PCR primer (Puro) | GTCAGGCACCGGGCTTGC |
| | | SEQ ID NO: 15 |
| HSP-525 | PCR primer (Puro-GFP) | GCGCAGATCTCTGCAGAGCTCGAGTGATCATGTGGAATGTGTGTCAGTT |
| | | AGG |
| | | SEQ ID NO: 16 |
| HSP-526 | PCR primer (Puro-GFP) | GCGCAGATCTGCGGCCGCTTTACTTGTACAGC |
| | | SEQ ID NO: 17 |

TABLE 3
Concentration results for Mu transposon constructs.
Kan/Neo-LoxP-Mu (2135 bp):

| | |
|---|---|
| Concentration of | 77.5 ng transposon DNA/μl = 0.055 pmol/μl |
| assembly reaction | |
| Final concentration | 705.1 ng transposon DNA/μl = 0.5 pmol/μl |
| | (9.1-fold increase in concentration) |

Kan/Neo-p15A-Mu (2795 bp):

| | |
|---|---|
| Concentration of | 101.6 ng transposon DNA/μl = 0.055 pmol/μl |
| assembly reaction | |
| Final concentration | 955.9 ng transposon DNA/μl = 0.515 pmol/μl |
| | (9.4-fold increase in concentration) |

Puro-eGFP-Mu (2065 bp):

| | |
|---|---|
| Concentration of | 74.5 ng transposon DNA/μl = 0.055 pmol/μl |
| assembly reaction | |
| Final concentration | 662 ng transposon DNA/μl = 0.49 pmol/μl |
| | (8.9-fold increase in concentration) |

### REFERENCES

Allet, B. (1979). Mu insertion duplicates a 5 base pair sequence at the host inserted site. Cell 1, 123-129.
Ausubel, F. M., Brent, R., Kingston, R. E., Moore, D. D., Seidman, J. G., Smith, J. A., and Struhl, K. (1989). Current protocols in molecular biology. John Wiley & Sons, New York, N.Y.
Baker, T. A., and Luo, L. (1994). Identification of residues in the Mu transposase essential for catalysis. Proc. Natl. Acad. Sci. U.S.A. 91, 6654-6658.
Baker, T. A., Mizuuchi, M., Savilahti, H., and Mizuuchi, K. (1993). Division of labor among monomers within the Mu transposase tetramer. Cell 74, 723-733.
Butterfield, Y. S., Marra, M. A., Asano, J. K., Chan, S. Y., Guin, R., Krzywinski, M. I., Lee, S. S., MacDonald, K. W., Mathewson, C. A., and Olson, T. E. et al. (2002). An efficient strategy for large-scale high-throughput transposon-mediated sequencing of cDNA clones. Nucleic Acids Res. 11, 2460-2468.
Chaconas, G., Lavoie, B. D., and Watson, M. A. (1996). DNA transposition: jumping gene machine, some assembly required. Curr. Biol. 7, 817-820.
Gropp M, Itsykson P, Singer O, Ben-Hur T, Reinhartz E, Galun E, Reubinoff B E. (2003) Stable genetic modification of human embryonic stem cells by lentiviral vectors. Mol. Ther. 7, 281-287
Haapa, S., Suomalainen, S., Eerikainen, S., Airaksinen, M., Paulin, L., and Savilahti, H. (1999a). An efficient DNA sequencing strategy based on the bacteriophage mu in vitro DNA transposition reaction. Genome Res. 3, 308-315.
Haapa, S., Taira, S., Heikkinen, E., and Savilahti, H. (1999b). An efficient and accurate integration of mini-Mu transposons in vitro: a general methodology for functional genetic analysis and molecular biology applications. Nucleic Acids Res. 13, 2777-2784.
Kahmann, R., and Kamp, D. (1979). Nucleotide sequences of the attachment sites of bacteriophage Mu DNA. Nature 5719, 247-250.
Kekarainen, T., Savilahti, H., and Valkonen, J. P. (2002). Functional genomics on potato virus A: virus genome-wide map of sites essential for virus propagation. Genome Res. 4, 584-594.
Lamberg, A., Nieminen, S., Qiao, M., and Savilahti, H. (2002). Efficient insertion mutagenesis strategy for bacterial genomes involving electroporation of in vitro-assembled DNA transposition complexes of bacteriophage mu. Appl. Environ. Microbiol. 2, 705-712.
Laurent, L. C., Olsen, M. N., Crowley, R. A., Savilahti, H., and Brown, P. O. (2000). Functional characterization of the human immunodeficiency virus type 1 genome by genetic footprinting. J. Virol. 6, 2760-2769.
Mikkola, M., Olsson, C., Palgi, J., Ustinov, J., Palomaki, T., Horelli-Kuitunen, N., Knuutila, S., Lundin, K., Otonkoski, T., and Tuuri, T. (2006). Distinct differentiation characteristics of individual human embryonic stem cell lines. BMC Dev. Biol. 6, 40.
Mizuuchi, K. (1992). Transpositional recombination: mechanistic insights from studies of mu and other elements. Annu. Rev. Biochem. 1011-1051.
Morgenstern, J. P. and Land, H. (1990) Advanced mammalian gene transfer: high titre retroviral vectors with multiple drug selection markers and a complementary helper-free packaging cell line. Nucleic Acids Res. 18, 3587-96
Pajunen, M. I., Pulliainen, A. T., Finne, J., and Savilahti, H. (2005). Generation of transposon insertion mutant libraries for Gram-positive bacteria by electroporation of phage Mu DNA transposition complexes. Microbiology 151, 1209-1218.
Poussu, E., Vihinen, M., Paulin, L. and Savilahti, H. (2004) Probing the α-complementing domain of E. coli β-galactosidase with use of an insertional pentapeptide mutagenesis strategy based on Mu in vitro DNA transposition. Proteins: Structure, Function, and Bioinformatics 54, 681-692
Sambrook, J. and Russell, d.W. (2001). Molecular cloning: a laboratory manual, 3^{rd} ed. Cold Spring Harbor Laboratory Press, Cold Spring Harbor, N.Y.
Savilahti, H. and Bamford D. H. (1993). Protein-primed DNA replication: role of inverted terminal repeats in the Escherichia coli bacteriophage PRD1 life cycle. J. Virol. 67(8), 4696-4703.
Savilahti, H., and Mizuuchi, K. (1996). Mu transpositional recombination: donor DNA cleavage and strand transfer in trans by the Mu transposase. Cell 2, 271-280.
Savilahti, H., Rice, P. A. and Mizuuchi, K. (1995) The phage Mu transpososome core: DNA requirements for assembly and function. EMBO J. 14, 4893-4903
Taira, S., Tuimala, J., Roine, E., Nurmiaho-Lassila, E. L., Savilahti, H., and Romantschuk, M. (1999). Mutational analysis of the Pseudomonas syringae pv. tomato hrpA gene encoding Hrp pilus subunit. Mol. Microbiol. 4, 737-744.
Takahashi K, Tanabe K, Ohnuki M, Narita M, Ichisaka T, Tomoda K, ym. Induction of pluripotent stem cells from adult human fibroblasts by defined factors. Cell 2007; 131:861-72.
Thomas, C. E., Ehrhardt, A. and Kay, M. A. (2003) Progress and problems with the use of viral vectors for gene therapy. Nature Reviews Genetics 4, 346-358.
Vilen, H., Eerikainen, S., Tornberg, J., Airaksinen, M. S., and Savilahti, H. (2001). Construction of gene-targeting vectors: a rapid Mu in vitro DNA transposition-based strategy generating null, potentially hypomorphic, and conditional alleles. Transgenic Res. 1, 69-80.
Vilen, H., Aalto, J-M., Kassinen, A., Paulin, L., and Savilahti, H. (2003). A direct transposon insertion tool for modification and functional analysis of viral genomes. J. Virol. 77, 123-134.
Wernig M, Meissner A, Foreman R, Brambrink T, Ku M, Hochedlinger K, ym. In vitro reprogramming of fibroblasts into a pluripotent ES-cell-like state. Nature 2007; 448:318-24.
Wiethoff, C. M. and Middaugh, C. R. (2003) Barriers to nonviral gene delivery. J Pharm Sci. 92, 203-17.
Yu J, Vodyanik M A, Smuga-Otto K, Antosiewicz-Bourget J, Frane J L, Tian S, ym. Induced pluripotent stem cell lines derived from human somatic cells. Science 2007; 318:1917-20.

1-13. (canceled)

14. A method for incorporating nucleic acid segments into cellular nucleic acid of an isolated human stem cell, the method comprising the step of:
delivering into the human stem cell an in vitro assembled Mu transposition complex that comprises (i) MuA transposases and (ii) a transposon segment that comprises a pair of Mu end sequences recognised and bound by MuA transposase and an insert sequence between said Mu end sequences.

15. The method according to claim 14, wherein said Mu transposition complex is delivered into the target cell by electroporation.

16. The method according to claim 14, wherein the nucleic acid segment is incorporated to a random or almost random position of the cellular nucleic acid of the target cell.

17. The method according to claim 14, wherein the nucleic acid segment is incorporated to a targeted position of the cellular nucleic acid of the target cell.

18. The method according to claim 14, wherein the target cell is a human ES cell or a human adult stem cell.

19. The method according to claim 14, wherein said insert sequence comprises a marker, which is selectable in human cells.

20. The method according to claim 14, wherein a concentrated fraction of Mu transposition complexes are delivered into the target cell.

21. The method according to claim 14 further comprising the step of incubating the target cells under conditions that promote transposition into the cellular nucleic acid.

22. A method for forming an insertion mutant library from a pool of human stem cells, the method comprising the steps of:
a) delivering into the human stem cell an in vitro assembled Mu transposition complex that comprises (i) MuA transposases and (ii) a transposon segment that comprises a pair of Mu end sequences recognised and bound by MuA transposase and an insert sequence with a selectable marker between said Mu end sequences, under conditions that allow integration of the transposon segment into the cellular nucleic acid; and
b) screening for cells that comprise the selectable marker.

23. Use of a kit comprising a concentrated fraction of Mu transposition complexes with a transposon segment that comprises a marker, which is selectable in human cells, for incorporating nucleic acid segments into cellular nucleic acid of an isolated human stem cell.

24. Use of the transposon nucleic acid comprising the sequence set forth in SEQ ID NO:1 in an in vitro assembled Mu transposition complex for incorporating nucleic acid segments into cellular nucleic acid of an isolated human stem cell.

25. Use of the transposon nucleic acid comprising the sequence set forth in SEQ ID NO:2 in an in vitro assembled Mu transposition complex for incorporating nucleic acid segments into cellular nucleic acid of an isolated human stem cell.
